(19) United States
(12) Patent Application Publication
(10) Pub. No.: US 2003/0223982 A1
(43) Pub. Date: Dec. 4, 2003

(54) Use of DNA repair enzymes as MMP 1 inhibitors

(76) Inventors: Kordula Schlotmann, Duesseldorf (DE); Thomas Foerster, Erkrath (DE); Dirk Petersohn, Koeln (DE); Marianne Waldmann-Laue, Monheim (DE)

Correspondence Address: HENKEL CORPORATION, 2500 RENAISSANCE BLVD, STE 200, GULPH MILLS, PA, 19406, US

(21) Appl. No.: 10/459,339

(22) Filed: Jun. 11, 2003

Related U.S. Application Data
(63) Continuation of application No. PCT/EP01/14514, filed on Dec. 11, 2001.

(30) Foreign Application Priority Data
Dec. 20, 2000 (DE) 100 63 433.8

(51) Int. Cl. A61K 38/47

(52) U.S. Cl. 424/94.61

(57) ABSTRACT

A method of using DNA repair enzymes in topical cosmetic or pharmaceutical compositions for the prevention of the ageing of human skin. In particular, the method prevents ageing effects caused by light. The method prevents the degradation of collagen within connective tissue, specifically within the dermis of the skin.

### CROSS REFERENCE TO RELATED APPLICATIONS

This application is a continuation under 35 USC 365(c) and 35 USC 120 of international application PCT/EP01/14514, filed on Dec. 11, 2001, the international application not being published in English. This application also claims priority under 35 USC 119 to DE 100 63 433.8, filed on Dec. 20, 2000.

### BACKGROUND OF THE INVENTION

This invention relates to the use of certain DNA repair enzymes as inhibitors of the collagen-degrading matrix metal proteinase 1 (MMP 1) in cosmetic or pharmaceutical compositions for preventing the ageing, particularly the light-induced ageing, of human skin.

Exposure to sunlight leads to changes in the biochemical equilibrium of the skin.

In the dermis and particularly in dermal fibroblasts, the UV component and infrared radiation (heat) lead through various mechanisms to the induction of the interstitial collagenase MMP 1, an enzyme which degrades the collagen components of the connective tissue. In the context of the present invention, the induction of the collagenase MMP 1 can be understood to mean both an increase in the quantity of this enzyme and an increase in its activity or both. MMP 1 separates the fibrillar triple-helix collagen at a particular point of the molecule. The triple helix divided into two parts dissolves and is made accessible to degradation by other collagenases. Macroscopically, the reduction in the quantity of collagen is reflected in a reduction in the elasticity of the skin and in the formation of wrinkles. Induction of the collagenase MMP 1 by UV radiation is regarded as the main reason for the macroscopic effects of skin ageing.

In the context of the invention, an MMP 1 inhibitor is a substance which

(a) inhibits the production of mRNA which codes the enzyme MMP 1 and hence reduces or prevents the expression of the enzyme and/or

(b) reduces activation of the enzyme MMP 1 and/or

(c) reduces the activity of the enzyme MMP 1.

Reducing the synthesis of MMP 1 and/or MMP 1 activity is therefore an important goal in the development of anti-ageing skin cosmetics, i.e. cosmetic products which counteract ageing of the skin. An ideal anti-ageing substance inhibits the expression of MMP 1, even in low concentrations. In addition, the substance should not be toxic to cells and should be stable in cosmetic and pharmaceutical formulations.

There are other matrix metal proteinases besides MMP 1 in the skin. Any reduction in the synthesis or activity of the other MMPs is not regarded as advantageous because they perform physiologically important functions.

The anti-ageing substances known from the prior art meet these requirements unsatisfactorily, if at all. WO 98/55075 claims triple combinations of a UV-A blocker, a UV-B blocker and an MMP inhibitor which counteract light-induced ageing of the skin. To be effective, the compositions have to be applied to the skin 7 to 48 hours before exposure to UV light. Retinoic acid (tretinoin) and retinol are preferred MMP inhibitors. Retinoids engage in the metabolism of the skin cells and, besides stimulating the proliferation and differentiation of the epidermal keratinocytes, increase the production of collagen by fibroblasts. In addition, retinol is said to reduce the formation of collagen-digesting enzymes (New Scientist 2031, 42-46, 1996). However, retinoic acid has teratogenous properties and can only be used in prescription pharmaceuticals. The use of retinol in cosmetic and pharmaceutical topical preparations is problematical for several reasons. Thus, retinol has a relatively high cell toxicity and, more particularly, phototoxicity and, accordingly, may only be used in low concentrations in compositions intended for human application. In addition, retinol is readily degraded by oxidation under the effect of heat and/or light and is difficult to stabilize in cosmetic and pharmaceutical formulations.

The problem addressed by the present invention was to remedy the deficiencies of the prior art and to provide more suitable preparations for the cosmetic treatment of sunlight-induced ageing of the skin. Another problem addressed by the invention was to provide compositions suitable for the pharmaceutical treatment of sunlight-induced ageing of the skin.

### SUMMARY OF THE INVENTION

It has now surprisingly been found that the enzymes photolyase and T4 endonuclease V inhibit the UV-induced expression of MMP 1 in the skin.

Photolyase and T4 endonuclease V, hereinafter referred to in short as “T4N5”, are already known in the prior art as so-called DNA repair enzymes. In the context of the invention, DNA repair is understood to be the cleavage or removal of UV-induced pyrimidine dimers from DNA.

“Pyrimidine dimer” is the name commonly used in the prior art for dimers which are formed photochemically, for example by UV-B rays, from certain pyrimidine bases of DNA. Although pyrimidine itself is not a DNA base, the term “pyrimidine dimer” is used in the following instead of the correct term “pyrimidine base dimer”. Dimerization at the pyrimidine base thymine takes place by dimerization of adjacent thymine units of a DNA strand to form a tricyclic compound. The dimerization product, a cis-syn cyclobutane dipyrimidine unit, can initiate errors in the transmission of the genetic code. The epidermal keratinocytes above all are affected by the formation of the pyrimidine derivatives.

Photolyase is the short name for deoxyribodipyrimidine photolyase or DNA photolyase, an enzyme with the classification number EC 4.1.99.3. Photolyase was found in lower eukaryotes, for example yeasts. It requires light in the 350-500 nm wavelength range to become activated. This light is absorbed by a chromophore group present in the photolyase molecule which subsequently transfers electrons to a second chromophore. By the further transfer of electrons, the cyclobutane dipyrimidine unit is split and the two original thymine bases are reformed. A particularly efficient photolyase comes from Anacystis nidulans, a phototrophic marine microorganism. The photolyase from A. nidulans has meanwhile been obtained in commercially relevant quantities from E. coli.

The enzyme T4 endonuclease V is produced by the denV gene of the bacteriophage T4 and belongs to the phosphodiesterases which hydrolyze the nucleic acids at the (5′-3′) bond. As an endonuclease, T4N5 attacks within the nucleic acid strand. In doing so, it selectively recognizes the DNA regions that are damaged by UV-induced pyrimidine dimers and excises them. New correct bases are incorporated by polymerases with the aid of the complementary strand as matrix and are linked by ligases to the original DNA strand. This excision repair mechanism is a dark reaction which does not require light activation. Although T4N5 is a prokaryote enzyme, it also acts on human cells. It can be industrially produced from E. coli strains that contain the denV gene.

An overview of important research results on DNA repair by photolyase and T4N5 is presented by D. Yarosh and E. Klein in Trends in Photochemistry & Photobiology 3,175-181, 1994.

DNA repair enzymes are an interesting active substance for cosmetic compositions. In the prior art, the preferred cosmetic compositions are sun protection preparations and after-sun products. The liposome encapsulation of T4N5 is described by Ceccoli et al. in J. Invest. Dermatol. 93, 190-194, 1989. The use of liposome-encapsulated T4N5 or photolyase in cosmetic preparations is described by Yarosh (U.S. Pat. No. 5,190,762; WO 94/14419 A1) and Gilchrest et al. (WO 94/17781 A1). Burmeister et al. (EP 0 707 844 A2) disclose compositions containing liposome-encapsulated combinations of DNA repair enzymes with tyrosine, tyrosine derivatives, vitamins or provitamins of vitamin groups A, C and E, glycoprotein complexes of copper, zinc or magnesium, forskolin, cyclic adenosine monophosphate (c-AMP), bioflavonoids or emulsifiers with an HB value of 10-14 and processes for the production of cosmetic tanning preparations and skin care products.

The fact that T4N5 promotes increased melanogenesis and can therefore be used in tanning preparations is described in EP 0 707 844 A2, WO 94/14419 A1 and WO 94/17781 A1. Photolyase has no effect on melanogenesis and may therefore be used in skin lightening products (S. H. Lee, KR 97032828 A). Liposome-encapsulated photolyase is commercially available, for example, as Photosome™ while liposome-encapsulated T4N5 is commercially available, for example, as Ultrasome™ from Applied Genetics, Freeport USA.

In the prior art, photolyase or T4N5 is only disclosed in connection with the repair of pyrimidine dimer-damaged DNA. In addition, for T4N5, there is the reference to an increase in melanogenesis. The known effects relate above all to the epidermis. The MMP 1-inhibiting effect, which relates above all to the dermis, is not known in the prior art. A hypothesis for the MMP 1-inhibiting effect of photolyase or T4N5 is presented in the following. In the case of UV damage to the DNA, an in-cell repair mechanism—transcription-coupled repair—is normally activated. This mechanism leads in the epidermis to an increase in the synthesis of the interleucines IL-1 and LI-6. The interleucines translocate into the dermis and bind at receptors to the fibroblasts. In response, collagen-degrading MMP 1 is synthesized by the fibroblasts. Surprisingly and unforeseeably to the expert, the DNA repair enzymes photolyase and T4N5 are evidently capable of healing UV-induced DNA damage before the in-cell transcription-coupled repair mechanism is activated and the causal chain for the UV-induced MMP 1 synthesis is set in motion.

The use of photolyase or T4N5 in accordance with the invention for inhibiting the expression of MMP 1 and for delaying the degradation of collagen is new. It opens up new applications in the cosmetic treatment of skin ageing which go beyond the known applications. MMP 1 inhibitors may be used with advantage anywhere in cosmetics where cosmetically desirable effects are associated with the inhibition of MMP 1. Accordingly, the use of photolyase or T4N5 is recommended, for example, in anti-wrinkle creams, especially for those areas of skin on the face, the neck or the hands that are constantly exposed to light. Concentrated creams, lotions, plasters and patches containing photolyase or T4N5 as MMP 1 inhibitors may be produced for the local treatment of wrinkles. T4N5 may even be used for wrinkle treatment after UV exposure on parts of the body that are normally seldom exposed to light, for example in creams and lotions for the whole body, because this enzyme does not require exposure to light of the treated regions to be activated. The DNA repair enzymes may be used in accordance with the invention both for preventive cosmetic treatment and also for delaying the macroscopic effects of skin ageing, especially the sunlight-induced ageing of human skin.

The skin treatment preparations according to the invention are suitable for preventing sunlight-induced ageing of the skin both in cases of sun exposure below the minimal erythemal dose (MED) and in cases of exposure above the MED. Accordingly, they are suitable both for preventive long-term treatment, their daily use affording the skin long-term protection, even in cases of minimal exposure to the sun, and for prevention against high exposure to sunlight. For the latter case in particular, the MMP 1-inhibiting compositions may be used both before and after exposure to the sun, i.e. in both cases the effect on the skin required in accordance with the invention is achieved. It is particularly advantageous that the MMP 1 inhibitors according to the invention prevent sunlight-induced ageing of the skin even when they are topically applied to the skin only a relatively short time before it is exposed to sunlight. A relatively short time in this context is understood in particular to be a time of one to five hours.

### DETAILED DESCRIPTION OF THE INVENTION

In a first embodiment, therefore, the invention relates to the use of DNA repair enzymes in cosmetic topical skin treatment compositions for inhibiting the light-induced degradation of collagen.

The present invention also relates to the use of DNA repair enzymes in cosmetic topical skin treatment compositions for inhibiting the expression or activity of the matrix metal proteinase MMP 1. The present invention further relates to the use of DNA repair enzymes for the production of pharmaceutical topical skin treatment compositions which inhibit the light-induced degradation of collagen and to the use of DNA repair enzymes for the production of pharmaceutical topical skin treatment compositions which inhibit the expression or activity of the matrix metal proteinase MMP 1.

In another embodiment, the present invention relates to the use of DNA repair enzymes in topical skin treatment compositions or anti-ageing compositions for reducing the loss of elasticity and the wrinkling of ageing skin. In a preferred embodiment, the DNA repair enzyme used in accordance with the invention is photolyase. Liposome-encapsulated photolyase is particularly preferred.

In another preferred embodiment, the DNA repair enzyme used in accordance with the invention is T4 endonuclease V. Liposome-encapsulated T4 endonuclease V is particularly preferred. The use in accordance with the invention of a mixture of photolyase and T4 endonuclease V, preferably in liposome-encapsulated form, is particularly preferred. In another preferred embodiment, the use according to the invention is preventive. In a preferred embodiment, the quantity of the DNA repair enzyme(s) used in accordance with the invention is between 1·10⁻⁶ and 5·10⁻²% by weight and more particularly between 1·10⁻⁵ and 1·10⁻²% by weight, based on the skin treatment composition as a whole.

The present invention also relates to a cosmetic or pharmaceutical skin treatment composition containing photolyase and/or T4 endonuclease V and, in addition, at least one substance selected from the vitamins, provitamins or vitamin precursors of the vitamin B group or derivatives thereof and derivatives of 2-furanone.

The vitamin B group or the vitamin B complex includes inter alia

vitamin B₁, trivial name thiamine, chemical name 3-[(4′-amino-2′-methyl-5′-pyimidinyl)-methyl]-5-(2-hydroxyethyl)-4-methyl thiazolium chloride. Thiamine hydrochloride is preferably used in quantities of 0.05 to 1% by weight, based on the composition as a whole.

vitamin B₂, trivial name riboflavin, chemical name 7,8-dimethyl-10-(1-D-ribityl)-benzo [g]pteridine-2,4(3H, 10H)-dione. In free form, riboflavin occurs, for example, in whey; other riboflavin derivatives can be isolated from bacteria and yeasts. A stereoisomer of riboflavin also suitable for the purposes of the invention is lyxoflavin which can be isolated from fish meal or liver and which carries a D-arabityl residue instead of D-ribityl. Riboflavin or its derivates are preferably used in quantities of 0.05 to 1% by weight, based on the composition as a whole.

Vitamin B₃. The compounds nicotinic acid and nicotinic acid amide (niacinamide) frequently go under this name. Nicotinic acid amide is preferred for the purposes of the invention and is preferably present in the compositions according to the invention in quantities of 0.05 to 1% by weight.

Vitamin B₅ (pantothenic acid and panthenol). Panthenol is preferably used. Derivatives of panthenol suitable for use in accordance with the invention are, in particular, the esters and ethers of panthenol and cationically derivatized panthenols. In another preferred embodiment of the invention, derivatives of 2-furanone with the following general structural formula:

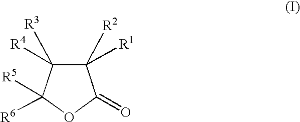

may also be used instead of or in addition to pantothenic acid or panthenol. Preferred 2-furanone derivatives are those in which the substituents R¹ to R⁶ independently of one another represent a hydrogen atom, a hydroxyl group, a methyl, methoxy, aminomethyl or hydroxymethyl group, a saturated or mono- or di-unsaturated, linear or branched C₂₋₄ hydrocarbon radical, a saturated or mono- or di-unsaturated, branched or linear mono-, di- or trihydroxy-C₂₋₄-hydrocarbon radical or a saturated or mono- or di-unsaturated, branched or linear mono-, di- or triamino-C₂₋₄-hydrocarbon radical. Other particularly preferred derivatives are the commercially available substances dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), all stereoisomers being expressly included. The 2-furanone derivative most particularly preferred for the purposes of the invention is pantolactone (dihydro-3-hydroxy-4,4-dimethyl-2(3H)-furanone), R¹ in formula (I) being a hydroxyl group, R² a hydrogen atom, R³ and R⁴ a methyl group and R⁵ and R⁶ a hydrogen atom.

The stereoisomer (R)-pantolactone is formed in the degradation of pantothenic acid.

The above-mentioned compounds of the vitamin B₅ type and the 2-furanone derivatives are preferably present in the compositions according to the invention in a total quantity of 0.05 to 10% by weight, based on the composition as a whole. Total quantities of 0.1 to 5% by weight are particularly preferred.

Vitamin B₆ which is not a single substance but rather derivatives of 5-hydroxymethyl-2-methylpyridin-3-ol known by the trivial names of pyridoxine, pyridoxamine and pyridoxal. Vitamin B₆ is preferably present in the compositions according to the invention in quantities of 0.0001 to 1.0% by weight and more particularly in quantities of 0.001 to 0.01% by weight.

Vitamin B₇ (biotin), also known as vitamin H or “skin vitamin”. Biotin is (3aS,4S,6aR)-2-oxohexahydrothienol[3,4-d]-imidazole-4-valeric acid. Biotin is preferably present in the compositions according to the invention in quantities of 0.0001 to 1.0% by weight and more particularly in quantities of 0.001 to 0.01% by weight.

Panthenol, pantolactone, nicotinic acid amide and biotin are most particularly preferred for the purposes of the invention.

The present invention also relates to a cosmetic or pharmaceutical skin treatment composition containing photolyase and/or T4 endonuclease V and also at least one plant extract. Plant extracts are normally prepared by extraction of the whole plant, but in some cases solely from flowers and/or leaves and/or seeds and/or other plant parts. Above all, extracts of the meristem, i.e. the divisable formative tissue of the plants, and special plants, such as green tea, hamamelis, chamomile, marigold, pansy, peony, aloe vera, horse chestnut, sage, willow bark, cinnamon tree, chrysanthemum, oak bark, stinging nettle, hops, burdock root, horse willow, hawthorn, lime blossom, almond, pine needle, sandalwood, juniper, coconut, kiwi, guava, lime, mango, apricot, wheat, melon, orange, grapefruit, avocado, rosemary, birch, beech shoots, mallow, lady's smock, yarrow, creeping thyme, thyme, balm, restharrow, hibiscus (althaea), mallow (Malva sylvestris), violet, black currant leaves, horseradish, cinquefoil, ginseng, ginger root and sweet potato are preferred for the purposes of the invention. Algal extracts may also be used with advantage. The algal extracts used in accordance with the invention come from green algae, brown algae, red algae or blue algae (cyanobacteria). The algae used for extraction may be both of natural origin and obtained by biotechnological processes and, if desired, may be modified in relation to the natural form. The organisms may be modified genetically, by growing or by cultivation in media enriched with selected nutrients. Preferred algal extracts come from seaweed, blue algae, from the green algae Codium tomentosum and from the brown algae Fucus vesiculosus. A particularly preferred algal extract comes from blue algae of the spirulina species cultivated in a magnesium-enriched medium.

Extracts of spirulina, green tea, aloe vera, meristem, hamamelis, apricot, marigold, guava, sweet potato, lime, mango, kiwi, cucumber, mallow, hibiscus and violet are particularly preferred. The compositions according to the invention may also contain mixtures of several, more particularly two, different plant extracts.

Suitable extractants for preparing the plant extracts mentioned are inter alia water, alcohols and mixtures thereof. Preferred alcohols are lower alcohols, such as ethanol and isopropanol, but especially polyhydric alcohols, such as ethylene glycol, propylene glycol and butylene glycol, which may be used either on their own or in the form of a mixture with water. Plant extracts based on water/propylene glycol in a ratio of 1:10 to 10:1 have proved to be particularly suitable. According to the invention, steam distillation is among the preferred extraction processes.

According to the invention, the plant extracts may be used both in pure form and in dilute form. If they are used in dilute form, they normally contain ca. 2 to 80% by weight active substance and, as solvent, the extractant or extractant mixture used in their preparation. Depending on the choice of extractant, it can be of advantage to stabilize the plant extract by addition of a solubilizer. Suitable solubilizers are, for example, ethoxylation products of optionally hydrogenated vegetable and animal oils. Preferred solubilizers are ethoxylated mono-, di- and triglycerides of C₈₋₂₂ fatty acids containing 4 to 50 ethylene oxide units, for example hydrogenated ethoxylated castor oil, olive oil ethoxylate, almond oil ethoxylate, mink oil ethoxylate, polyoxyethylene glycol caprylic/capric acid glycerides, polyoxyethylene glycerol monolaurate and polyoxyethylene glycol coconut fatty acid glycerides.

In another preferred embodiment, mixtures of several, more particularly two, different plant extracts are used in the compositions according to the invention.

So far as the plant extracts usable in accordance with the invention are concerned, particular reference is made to the extracts that are listed in the Table beginning on page 44 of the 3rd Edition of the Leitfaden zur Inhaltsstoffdeklaration kosmetischer Mittel, published by the Industrieverband Körperpflege und Waschmittel e.V. (IKW), Frankfurt.

The present invention also relates to a cosmetic or pharmaceutical skin treatment composition containing photolyase and/or T4 endonuclease V and at least one other MMP 1-inhibiting substance selected from propyl gallate, precocenes, 6-hydroxy-7-methoxy-2,2-dimethyl-1 (2H)-benzopyran, 3,4-dihydro-6-hydroxy-7-methoxy-2,2-dimethyl-1 (2H)-benzopyran (commercially obtainable as Lipochroman 6® from Lipotec S.A.) and mixtures thereof. Precocenes are chromene derivatives occurring in plants which are known as hormones (The Merck Index, 12th Edition, Merck & Co. 1996). The MMP 1-inhibiting effect of these substances is described in DE 10016016 A1. They are used in quantities of 0.1 to 5 and preferably 0.5 to 2% by weight, based on the composition as a whole.

In one particularly preferred embodiment, the skin treatment compositions according to the invention additionally contain at least one ester of retinol (vitamin A₁) with a C₂₋₁₈ carboxylic acid. Preferred retinol esters are retinyl acetate and retinyl palmitate. Retinyl palmitate is particularly preferred. The retinol esters are used in quantities of 0.1 to 5% by weight and preferably in quantities of 0.5 to 2% by weight, based on the composition as a whole.

In another preferred embodiment, the skin treatment compositions according to the invention contain at least one surfactant as emulsifier or dispersant. Emulsifiers promote the formation at the phase interface of water-stable or oil-stable adsorption layers which protect the dispersed droplets against coalescence and thus stabilize the emulsion. Accordingly, emulsifiers like surfactants are made up of a hydrophobic and a hydrophilic molecule part. Hydrophilic emulsifiers preferentially form o/w emulsions while hydrophobic emulsifiers preferentially form w/o emulsions. W/o emulsions stabilized without hydrophilic emulsifiers are disclosed in DE 19816665 A1 and DE 19801593 A1. An emulsion is understood to be a droplet like dispersion of one liquid in another liquid using energy to create stabilizing phase interfaces by means of surfactants. The choice of these emulsifying surfactants or emulsifiers is governed by the substances to be dispersed, the particular outer phase and the droplet fineness of the emulsion.

The following are examples of emulsifiers which may be used in accordance with the invention:

products of the addition of 4 to 30 mol ethylene oxide and/or 0 to 5 mol propylene oxide onto linear C₈₋₂₂ fatty alcohols, onto C₁₂₋₂₂ fatty acids and onto C₈₋₁₅ alkyl phenols,

C₁₂₋₂₂ fatty acid mono- and diesters of addition products of 1 to 30 mol ethylene oxide onto C₃₋₆ polyols, more particularly glycerol,

ethylene oxide and polyglycerol addition products onto methyl glucoside/fatty acid esters, fatty acid alkanolamides and fatty acid glucamides,

C₈₋₂₂ alkyl mono- and oligoglycosides and ethoxylated analogs thereof, preferred degrees of oligomerization being 1.1 to 5 and more particularly 1.2 to 2.0 and glucose being preferred as the sugar component,

mixtures of alkyl (oligo)glucosides and fatty alcohols, for example the commercially available product Montanov® 68,

addition products of 5 to 60 mol ethylene oxide onto castor oil and hydrogenated castor oil,

partial esters of polyols containing 3 to 6 carbon atoms with saturated C₈₋₂₂ fatty acids,

sterols (sterins). Sterols are understood to be a group of steroids which carry a hydroxyl group at carbon atom 3 of the steroid skeleton and which are isolated both from animal tissue (zoosterols) and from vegetable fats (phytosterols). Examples of zoosterols are cholesterol and lanosterol. Examples of suitable phytosterols are beta-sitosterol, stigmasterol, campesterol and ergosterol. Sterols are also isolated from fungi and yeasts (so-called mycosterols).

phospholipids, above all the glucose phospholipids, which are obtained, for example, as lecithins or phosphatidyl cholines from, for example, egg yolk or plant seeds (for example soya beans),

fatty acid esters of sugars and sugar alcohols, such as sorbitol,

polyglycerols and polyglycerol derivatives, preferably polyglyceryl-2-dipolyhydroxystearate (commercial product Dehymuls® PGPH) and polyglyceryl-3-diisostearate (commercial product Lameform® TGI),

linear and branched C₆₋₃₀ fatty acids and Na, K, ammonium, Ca, Mg and Zn salts thereof.

The compositions according to the invention contain the emulsifiers in quantities of preferably 0.1 to 25% by weight and more particularly 0.5 to 15% by weight, based on the composition as a whole.

In one particularly preferred embodiment, at least one nonionic emulsifier with an HLB value of 8 or lower is present (for a definition of HLB value, see Römpp-Lexikon Chemie (Eds.: J. Falbe, M. Regitz), 10th Edition, Georg Thieme Verlag Stuttgart/New York (1997), page 1764). Suitable emulsifiers are, for example, compounds corresponding to the general formula R¹—O—R², where R¹ is a primary linear alkyl, alkenyl or acyl group containing 20 to 30 carbon atoms and R² is hydrogen, a group with the formula —(CₙH₂ₙO)ₓ—H with x=1 or 2 and n=2-4 or a polyhydroxyalkyl group containing 4 to 6 carbon atoms and 2 to 5 hydroxyl groups. A particularly preferred emulsifier with the formula R¹—O—R² is a behenyl or erucyl derivative in which R¹ is a linear, terminally substituted alkyl, alkenyl or acyl group containing 22 carbon atoms.

Other particularly suitable emulsifiers with an HLB value of 8 or lower are the addition products of 1 or 2 mol ethylene oxide or propylene oxide onto behenyl alcohol, erucyl alcohol, arachidyl alcohol or even onto behenic acid or erucic acid. Other preferred emulsifiers are the monoesters of C₁₆₋₃₀ fatty acids with polyols such as, for example, pentaerythritol, trimethylolpropane, diglycerol, sorbitol, glucose and methyl glucose. Examples of such products are, for example, sorbitan monobehenate or pentaerythritol monoerucate.

In another particularly preferred embodiment, at least one ionic emulsifier selected from anionic, zwitterionic, ampholytic and cationic emulsifiers is present. Preferred anionic emulsifiers are alkyl sulfates, alkyl polyglycol ether sulfates and ether carboxylic acids containing 10 to 18 carbon atoms in the alkyl group and up to 12 glycolether groups in the molecule, sulfosuccinic acid mono- and dialkyl esters containing 8 to 18 carbon atoms in the alkyl group and sulfosuccinic acid monoalkyl polyoxyethyl esters containing 8 to 18 carbon atoms in the alkyl group and 1 to 6 oxyethyl groups, monoglyceride sulfates, alkyl and alkenyl ether phosphates and protein fatty acid condensates. Zwitterionic emulsifiers carry at least one quaternary ammonium group and at least one —COO⁻ or —SO₃⁻ group in the molecule. Particularly suitable zwitterionic emulsifiers are the so-called betaines, such as the N-alkyl-N,N-dimethyl ammonium glycinates, N-acylaminopropyl-N,N-dimethyl ammonium glycinates and 2-alkyl-3-carboxymethyl-3-hydroxyethylimidazolines containing 8 to 18 carbon atoms in the alkyl or acyl group and cocoacylaminoethyl hydroxyethyl carboxymethyl glycinate.

In addition to a C₈₋₂₄ alkyl or acyl group, ampholytic emulsifiers contain at least one free amino group and at least one —COOH or —SO₃H group in the molecule and are capable of forming inner salts. Examples of suitable ampholytic emulsifiers are N-alkyl glycines, N-alkylaminopropionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 24 carbon atoms in the alkyl group.

The ionic emulsifiers are present in a quantity of 0.01 to 5% by weight, preferably in a quantity of 0.05 to 3% by weight and more particularly in a quantity of 0.1 to 1% by weight, based on the composition as a whole.

In another preferred embodiment, the skin treatment compositions according to the invention contain at least one organic or mineral or modified mineral light filters. The light filters are substances which are liquid or crystalline at room temperature and which are capable of absorbing ultraviolet radiation and of releasing the energy absorbed in the form of longer-wave radiation, for example heat. There are UVA and UVB filters. The UVA and UVB filters may be used both individually and in the form of mixtures. The use of filter mixtures is particularly preferred for the purposes of the invention.

The organic UV filters used in accordance with the invention are selected from the derivatives of dibenzoyl methane, cinnamic acid esters, diphenyl acrylic acid esters, benzophenone, camphor, p-aminobenzoic acid esters, o-aminobenzoic acid esters, salicylic acid esters, benzimidazoles, 1,3,5-triazines, monomeric and oligomeric 4,4-diarylbutadiene carboxylic acid esters and carboxylic acid amides, ketotricyclo(5.2.1.0)decane, benzal malonic acid esters and mixtures of the components mentioned. The organic UV filters may be oil-soluble or water-soluble. According to the invention, particularly preferred oil-soluble UV filters are 1-(4-tert.butylphenyl)-3-(4′-methoxyphenyl)-propane-1,3-dione (Parsol® 1789), 1-phenyl-3-(4′-isopropylphenyl)-propane-1,3-dione, 3-(4′-methylbenzylidene)-D,L-camphor, 4-(dimethylamino)-benzoic acid-2-ethylhexyl ester, 4-(dimethylamino)-benzoic acid-2-octyl ester, 4-(dimethylamino)-benzoic acid amyl ester, 4-methoxycinnamic acid-2-ethylhexyl ester, 4-methoxycinnamic acid propyl ester, 4-methoxycinnamic acid isopentyl ester, 2-cyano-3,3-phenylcinnamic acid-2-ethylhexyl ester (Octocrylene), salicylic acid-2-ethylhexyl ester, salicylic acid-4-isopropylbenzyl ester, salicylic acid homomenthyl ester (3,3,5-trimethylcyclohexyl salicylate), 2-hydroxy-4-methoxybenzophenone, 2-hydroxy-4-methoxy-4′-methylbenzophenone, 2,2′-dihydroxy-4-methoxybenzophenone, 4-methoxybenzmalonic acid di-2-ethylhexyl ester, 2,4,6-trianilino-(p-carbo-2′-ethyl-1′-hexyloxy)-1,3,5-triazine (Octyl Triazone) and Dioctyl Butamido Triazine (Uvasorb® HEB) and mixtures of the components mentioned.

Preferred water-soluble UV filters are 2-phenylbenzimidazole-5-sulfonic acid and alkali metal, alkaline earth metal, ammonium, alkylammonium, alkanolammonium and glucammonium salts thereof, sulfonic acid derivatives of benzophenones, preferably 2-hydroxy-4-methoxybenzophenone-5-sulfonic acid and salts thereof, sulfonic acid derivatives of 3-benzylidene camphor such as, for example, 4-(2-oxo-3-bornylidenemethyl)-benzenesulfonic acid and 2-methyl-5-(2-oxo-3-bornylidene)-sulfonic acid and salts thereof.

The inorganic UV protection pigments preferably used in accordance with the invention are finely disperse metal oxides and metal salts, for example titanium dioxide, zinc oxide, iron oxide, aluminium oxide, cerium oxide, zirconium oxide, silicate (talcum), barium sulfate and zinc stearate. The particles should have an average diameter of less than 100 nm, preferably from 5 to 50 nm and more preferably from 15 to 30 nm. They may be spherical in shape although ellipsoidal particles or other non-spherical particles may also be used. The pigments may also be surface-treated, i.e. hydrophilicized or hydrophobicized. Typical examples are coated titanium dioxides such as, for example, Titandioxid T 805 (Degussa) or Eusolex© T2000 (Merck). Suitable hydrophobic coating materials are, above all, silicones and particularly trialkoxyoctyl silanes or simethicones. So-called micro- or nanopigments are preferably used in sun protection products. Micronized zinc oxide is preferably used.

In addition, it has proved to be of particular advantage for the skin treatment compositions according to the invention to contain at least one protein hydrolyzate or a derivative thereof. Both vegetable and animal protein hydrolyzates may be used in accordance with the invention. Animal protein hydrolyzates are, for example, elastin, collagen, keratin, silk and milk protein hydrolyzates which may also be present in the form of salts. Vegetable protein hydrolyzates, for example soya, wheat, almond, pea, potato and rice protein hydrolyzates, are preferred for the purposes of the invention. Corresponding commercial products are, for example, DiaMin® (Diamalt), Gluadin® (Cognis), Lexein® (Inolex) and Crotein® (Croda).

Instead of the protein hydrolyzates, it is possible to use on the one hand otherwise obtained amino acid mixtures and, on the other hand, individual amino acids and physiologically compatible salts thereof. Amino acids preferred for the purposes of the invention include glycine, serine, threonine, cysteine, asparagine, glutamine, pyroglutamic acid, alanine, valine, leucine, isoleucine, proline, tryptophane, phenylalanine, methionine, aspartic acid, glutamic acid, lysine, arginine and histidine and also the zinc salts and the acid addition salts of the amino acids mentioned.

Derivatives of the protein hydrolyzates, for example in the form of their fatty acid condensation products, may also be used. Corresponding commercial products are, for example, Lamepon® (Cognis), Gluadin® (Cognis), Lexein® (Inolex), Crolastin® or Crotein® (Croda).

Cationized protein hydrolyzates may also be used in accordance with the invention, the basic protein hydrolyzate emanating from plants, marine organisms or biotechnologically obtained protein hydrolyzates. Cationic protein hydrolyzates of which the basic protein component has a molecular weight of 100 to 25,000 dalton and preferably 250 to 5,000 dalton are preferred. Cationic protein hydrolyzates are also understood to include quaternized amino acids and mixtures thereof. In addition, the cationic protein hydrolyzates may also be further derivatized. Typical examples of cationic protein hydrolyzates and derivatives used in accordance with the invention include some of the commercially obtainable products mentioned by their INCI names in the “International Cosmetic Ingredient Dictionary and Handbook” (Seventh Edition 1997, The Cosmetic, Toiletry and Fragrance Association 1101 17th Street, N.W., Suite 300, Washington, D.C. 20036-4702): Cocodimonium Hydroxypropyl Hydrolyzed Collagen, Cocodimonium Hydroxypropyl Hydrolyzed Casein, Steardimonium Hydroxypropyl Hydrolyzed Collagen, Steardimonium Hydroxypropyl Hydrolyzed Hair Keratin, Lauryldimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin. The cationic protein hydrolyzates and derivatives based on vegetable raw materials are most particularly preferred.

The protein hydrolyzates and their derivatives or the amino acids and their derivatives are present in the compositions according to the invention in quantities of 0.01 to 10% by weight, based on the composition as a whole. Quantities of 0.1 to 5% by weight and more particularly 0.1 to 3% by weight are particularly preferred.

In another advantageous embodiment, the skin treatment compositions according to the invention contain at least one mono-, oligo- or polysaccharide or derivative thereof.

Monosaccharides suitable for the purposes of the invention are, for example, glucose, fructose, galactose, arabinose, ribose, xylose, lyxose, allose, altrose, mannose, gulose, idose and talose, the deoxy sugars fucose and rhamnose and aminosugars such as, for example, glucosamine or galactosamine. Glucose, fructose, galactose, arabinose and fucose are preferred; glucose is particularly preferred.

Oligosaccharides suitable for the purposes of the invention are made up of two to ten monosaccharide units, for example sucrose, lactose or trehalose. A particularly preferred oligosaccharide is sucrose. The use of honey, which mainly contains glucose and sucrose, is also particularly preferred.

Polysaccharides suitable for the purposes of the invention are made up of more than ten monosaccharide units. Preferred polysaccharides are the starches made up of α-D-glucose units and starch degradation products, such as amylose, amylopectin and dextrins. According to the invention, chemically and/or thermally modified starches, for example hydroxypropyl starch phosphate, dihydroxypropyl distarch phosphate or the commercial products Dry Flo®, are particularly advantageous. Dextrans and dextran derivatives, for example dextran sulfate, are also preferred, as are nonionic cellulose derivatives, such as methyl cellulose, hydroxypropyl cellulose or hydroxyethyl cellulose, and cationic cellulose derivatives, for example the commercial products Celquat® and Polymer JR® and preferably Celquat® H 100, Celquat® L 200 and Polymer JR® 400 (Polyquaternium-10) and Polyquaternium-24. Other preferred examples are polysaccharides of fucose units, for example the commercial product Fucogel®). The polysaccharides made up of aminosugar units, more particularly chitins and deacetylated chitin derivatives, the chitosans, and mucopolysaccharides are particularly preferred. Mucopolysaccharides preferred for the purposes of the invention include hyaluronic acid and derivatives thereof, for example sodium hyaluronate and dimethylsilanohyaluronate, and chondroitin and derivatives thereof, for example chondroitin sulfate.

In one particularly advantageous embodiment, the skin treatment compositions according to the invention contain at least one film-forming, emulsion-stabilizing, thickening or adhesive polymer selected from natural and synthetic polymers which may be cationic, anionic, amphoterically charged or nonionic.

Cationic, anionic and nonionic polymers are preferred for the purposes of the invention. Among the cationic polymers, polysiloxanes containing quaternized groups, for example the commercial products Q2-7224 (Dow Corning), Dow Corning® 929 Emulsion (containing amodimethicone), SM-2059 (General Electric), SLM-55067 (Wacker) and Abil®-Quat 3270 and 3272 (Th. Goldschmidt), are preferred.

Preferred anionic polymers which can support the effect of the active substance used in accordance with the invention contain carboxylate and/or sulfonate groups and, as monomers, acrylic acid, methacrylic acid, crotonic acid, maleic anhydride and 2-acrylamido-2-methylpropanesulfonic acid for example. The acidic groups may be completely or partly present as sodium, potassium, ammonium, mono- or triethanolammonium salt. Preferred monomers are 2-acrylamido-2-methylpropanesulfonic acid and acrylic acid. Most particularly preferred anionic polymers contain 2-acrylamido-2-methylpropanesulfonic acid as sole monomer or comonomer, the sulfonic acid group being completely or partly present in salt form. In this embodiment, copolymers of at least one anionic monomer and at least one nonionic monomer are preferably used. So far as the anionic monomers are concerned, reference is made to the substances mentioned above. Preferred nonionic monomers are acrylamide, methacrylamide, acrylates, methacrylates, vinyl pyrrolidone, vinyl ethers and vinyl esters. Preferred anionic copolymers are acrylic acid/acrylamide copolymers and, in particular, polyacrylamide copolymers with monomers containing sulfonic acid groups. A particularly preferred anionic copolymer consists of 70 to 55 mol-% acrylamide and 30 to 45 mol-% 2-acrylamido-2-methyl-propanesulfonic acid, the sulfonic acid groups being completely or partly present as sodium, potassium, ammonium, mono- or triethanolammonium salt. This copolymer may also be crosslinked, preferred crosslinking agents being polyolefinically unsaturated compounds, such as tetraallyloxyethane, allyl sucrose, allyl pentaerythritol and methylene bis-acrylamide. One such polymer is present in the commercial product Sepigel® 305 of SEPPIC. The use of this compound has proved to be particularly advantageous for the purposes of the invention. The sodium acryloyl dimethyl taurate copolymers marketed as Simulgel® 600 in the form of a compound with isohexadecane and Polysorbate-80 have proved to be particularly effective for the purposes of the invention.

Other particularly preferred anionic homo- and copolymers are uncrosslinked and crosslinked polyacrylic acids. Allyl ethers of pentaerythritol, sucrose and propylene can be preferred crosslinking agents. Compounds such as these are, for example, the commercial products Carbopol®. Of a particularly preferred anionic copolymer, 80 to 98% contains an unsaturated, optionally substituted C₃₋₆ carboxylic acid or anhydride and 2 to 20% optionally substituted acrylates of saturated C₁₀₋₃₀ carboxylic acids, the copolymer optionally being crosslinked with the crosslinking agents mentioned above. Corresponding commercial products are Pemulen® and the Carbopol® types 954, 980, 1342 and ETD 2020 (ex B. F. Goodrich).

Suitable nonionic polymers are, for example, polyvinyl alcohols which may be partly saponified, for example the commercial products Mowiol®, and vinyl pyrrolidone/vinyl ether copolymers and polyvinyl pyrrolidones marketed, for example, under the name of Luviskol® (BASF).

In another preferred embodiment of the invention, the effect of the compositions according to the invention can be further optimized by fatty compounds. The following are examples of suitable fatty compounds:

Vegetable oils, such as sunflower oil, olive oil, soya oil, rapeseed oil, almond oil, jojoba oil, orange oil, wheatgerm oil, peach kernel oil and the liquid fractions of coconut oil.

Liquid paraffin oils, isoparaffin oils and synthetic hydrocarbons, for example 1,3-di-(2-ethylhexyl)-cyclohexane (Cetiol® S) or polydecene.

Di-n-alkylethers containing a total of 12 to 36 and more particularly 12 to 24 carbon atoms, for example di-n-octylether (Cetiol® OE), di-n-hexyl-n-octyl ether and n-octyl-n-decylether.

Fatty acids, particularly linear and/or branched, saturated and/or unsaturated C₈₋₃₀ fatty acids. C₁₀₋₂₂ fatty acids are preferred. Examples include the isostearic and isopalmitic acids, such as the fatty acids marketed under the name of Edenor®. Other typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidonic acid, gadoleic acid, behenic acid and erucic acid and technical mixtures thereof. Normally, the fatty acid cuts obtainable from coconut oil or palm oil are particularly preferred; the use of stearic acid is particularly preferred.

fatty alcohols, particularly saturated, mono- or polyunsaturated, branched or unbranched fatty alcohols containing 6 to 30, preferably 10 to 22 and more particularly 12 to 22 carbon atoms. Corresponding fatty alcohols suitable for use in accordance with the invention are, for example, decanol, octanol, octenol, dodecenol, decenol, octadienol, dodecadienol, decadienol, oleyl alcohol, erucyl alcohol, ricinolyl alcohol, stearyl alcohol, isostearyl alcohol cetyl alcohol, lauryl alcohol, myristyl alcohol, arachidyl alcohol, capryl alcohol, capric alcohol, linoleyl alcohol, linolenyl alcohol and behenyl alcohol, and Guerbet alcohols thereof, for example 2-ethyl hexanol, this list being intended to be purely exemplary with no limiting character.

Ester oils, i.e. esters of C₆₋₃₀ fatty acids with C₂₋₃₀ fatty alcohols. The monoesters of the fatty acids with C₂₋₂₄ alcohols are preferred. The alcohol and acid components of the ester oils may be selected from the substances mentioned above. Isopropyl myristate, isononanoic acid C₁₆₋₁₈ alkyl ester, 2-ethylhexyl palmitate, stearic acid-2-ethylhexyl ester, cetyl oleate, glycerol tricaprylate, coconut fatty alcohol caprate/caprylate, n-butylstearate, oleyl erucate, isopropyl palmitate, oleyl oleate, lauric acid hexyl ester, di-n-butyl adipate, myristyl myristate, Cetearyl Isononanoate and oleic acid decyl ester are particularly preferred for the purposes of the invention.

Hydroxycarboxylic alkyl esters, the full esters of glycolic acid, lactic acid, malic acid, tartaric acid or citric acid being preferred although esters of β-hydroxypropionic acid, tartronic acid, D-gluconic acid, saccharic acid, mucic acid or glucuronic acid are also suitable and the esters of C₁₂₋₁₅ fatty alcohols, for example the commercial products Cosmacol® of EniChem, Augusta Industriale, are particularly preferred.

Dicarboxylic acid esters, such as di-n-butyl adipate, di-(2-ethylhexyl)-adipate, di-(2-ethylhexyl)-succinate and diisotridecyl azelaate, and diol esters, such as ethylene glycol dioleate, ethylene glycol diisotridecanoate, propylene glycol di-(2-ethylhexanoate), propylene glycol diisostearate, propylene glycol dipelargonate, butanediol diisostearate, neopentyl glycol dicaprylate.

Symmetrical, nonsymmetrical or cyclic esters of carbonic acid with fatty alcohols, for example glycerol carbonate or dicaprylyl carbonate (Cetiol® CC).

Mono-, di- and trifatty acid esters of saturated and/or unsaturated linear and/or branched fatty acids with glycerol, for example Monomuls® 90-O18, Monomuls® 90-L12 or Cutina® MD.

Waxes, particularly insect waxes, such as beeswax and bumblebee wax, vegetable waxes, such as candelilla wax and carnauba wax, fruit waxes, ozocerite, microwax, ceresin, paraffin, triglycerides of saturated and optionally hydroxylated C₁₆₋₃₀ fatty acids such as, for example, hydrogenated triglyceride fats (hydrogenated palm oil, hydrogenated coconut oil, hydrogenated castor oil), glyceryl tribehenate or glyceryl tri-12-hydroxystearate, synthetic full esters of fatty acids and glycols (for example Syncrowachs®) or C₂₋₆ polyols, esters of optionally hydroxylated C₂₋₄ carboxylic acids with lanolin alcohols and C₁₂₋₁₈ fatty alcohols, cholesterol or lanosterol esters of C₁₀₋₃₀ fatty acids, ethoxylated C₁₂₋₂₀ fatty acid glycol esters, fatty acid monoalkanolamides with a C₁₂₋₂₂ acyl group and a C₂₋₄ alkanol group, synthetic fatty acid/fatty alcohol esters, for example stearyl stearate or cetyl palmitate and ester waxes of natural fatty acids and synthetic C₂₀₋₄₀ fatty alcohols (INCI name C20-40 Alkyl Stearate).

Silicone compounds selected from decamethyl cyclopentasiloxane, dodecamethyl cyclohexasiloxane and silicone polymers which may optionally be crosslinked, for example polydialkyl siloxanes, polyalkylaryl siloxanes, ethoxylated polydialkyl siloxanes, preferably the substances with the INCI name of Dimethicone Copolyol, and polydialkyl siloxanes containing amine and/or hydroxy groups.

The fatty compounds are used in quantities of 0.1 to 50% by weight, preferably in quantities of 0.1 to 20% by weight and more particularly in quantities of 0.1 to 15% by weight, based on the composition as a whole.

The compositions according to the invention may contain other active substances, auxiliaries and additives, for example:

vitamins, provitamins and vitamin precursors from the groups A, C, E and F, more particularly 3,4-didehydroretinol (vitamin A₂), β-carotene (provitamin of vitamin A₁), ascorbic acid (vitamin C) and the palmitic acid esters, glucosides or phosphates of ascorbic acid, tocopherols, more particularly α-tocopherol and its esters, for example the acetate, the nicotinate, the phosphate and the succinate; also vitamin F, i.e. essential fatty acids, particularly linoleic acid, linolenic acid and arachidonic acid;

allantoin;

bisabolol,

antioxidants, for example imidazoles (for example urocanic acid) and derivatives thereof, peptides, such as D,L-carnosine, D-carnosine, L-carnosine and derivatives thereof (for example anserine), chlorogenic acid and derivatives thereof, liponic acid and derivatives thereof (for example dihydroliponic acid), aurothioglucose, propylthiouracil and other thiols (for example thioredoxine, glutathione, cysteine, cystine, cysteamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, the coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, catalase, Superoxid-Dismutase, zinc and zinc derivatives (for example ZnO, ZnSO₄), selenium and derivatives thereof (for example selenium methionine), stilbenes and derivatives thereof (for example stilbene oxide, trans-stilbene oxide) and derivatives of these active substances suitable as antioxidants (salts, esters, ethers, sugars, nucleotides, nucleosides, peptides and lipids);

ceramides and pseudoceramides,

triterpenes, more particularly triterpenoic acids, such as ursolic acid, rosmaric acid, betulinic acid, boswelilic acid and bryonilic acid,

monomeric catechols, more particularly catechol and epicatechol, leucoanthocyanidines, catechol polymers (catechol tannins) and gallotannins,

thickeners, for example gelatin, vegetable gums, such as agar agar, guar gum, alginates, xanthan gum, gum arabic, karaya gum or locust beam gum, natural and synthetic clays and layer silicates, for example betonite, hectorite, montmorillonite or Laponite®, fully synthetic hydrocolloids such as, for example, polyvinyl alcohol, and also Ca, Mg or Zn soaps of fatty acids,

vegetable glycosides,

structurants, such as maleic acid and lactic acid,

dimethyl isosorbide,

alpha-, beta- and gamma-cyclodextrins, more particularly for stabilizing retinol,

solvents, swelling and penetration agents, such as ethanol, isopropanol, ethylene glycol, propylene glycol, propylene glycol monoethylether, glycerol and diethylene glycol, carbonates, hydrogen carbonates, guanidines, ureas and primary, secondary and tertiary phosphates,

perfume oils, pigments and dyes for coloring the composition,

pH adjusters, for example α- and β-hydroxycarboxylic acids,

complexing agents, such as EDTA, NTA, β-alanine diacetic acid and phosphonic acids,

opacifiers, such as latex, styrene/PVP and styrene/acylamide copolymers,

pearlizers, such as ethylene glycol mono- and distearate and PEG-3-distearate,

propellents, such as propane/butane mixtures, N₂O, dimethylether, CO₂ and air.

The skin treatment compositions according to the invention are advantageously present in the form of a liquid or solid oil-in-water emulsion, water-in-oil emulsion, multiple emulsion, microemulsion, PIT emulsion or Pickering emulsion, a hydrogel, a lipogel, a single-phase or multiphase solution, a foam, a powder or a mixture containing at least one polymer suitable as a medicinal adhesive. The compositions may also be administered in water-free form, for example as an oil or balm. In this case, the carrier may be a vegetable or animal oil, a mineral oil, a synthetic oil or a mixture of such oils.

In one particular embodiment, the compositions according to the invention are formulated as a microemulsion. Besides the thermodynamically stable microemulsions, microemulsions in the context of the invention are also understood to include so-called PIT emulsions. PIT emulsions are systems containing the three components water, oil and emulsifier which are present at room temperature as an oil-in-water emulsion. On heating, these systems form microemulsions in a certain temperature range (known as the phase inversion temperature or PIT) and, on further heating, change into water-in-oil (w/o) emulsions. On cooling, o/w emulsions are re-formed, but are present—even at room temperature—as microemulsions or as very fine-particle emulsions with a mean particle diameter below 400 nm and more particularly in the 100-300 nm range. Microemulsions or PIT emulsions with a mean particle diameter of about 200 nm can be preferred for the purposes of the invention. Particulars of these PIT emulsions can be found, for example, in the journal Angew. Chem. 97, 655-669 (1985).

The following Examples are intended to illustrate the invention without limiting it in any way.

### EXAMPLES

1. Studies of Multilayer Skin Models

The effect of liposome-encapsulated DNA repair enzymes on the inhibition of MMP 1 was investigated using a multilayer in vitro skin model. The skin model is a human skin equivalent which consists of a dermis with fibroblasts and an epidermis of keratinocytes.

This multilayer structure is formed in a special cultivation process. Dermal equivalents (DEs) were first produced by pipetting a suspension of 2×10⁵/cm² fibroblasts of human foreskin in a culture medium onto a matrix consisting of chitosan, collagen and glycosaminoglycans (matrix described in Collombel, C. et al.: Biomaterials with a base of collagen, chitosane and glycosaminoglycans, process for preparing them and their application in human medicine, U.S. Pat. No. 5,166,187). The culture medium consisted of Dulbecco's Modified Eagle's Medium (DMEM) supplemented with 10% fetal calf serum (FCS), 25 μg/ml gentamycin, 100 Ul/ml penicillin, 1 μg/ml amphotericin B, 50 μg/ml sodium ascorbate and 4 mM L-glutamine. The dermal equivalents were incubated in this medium for 14 days at 37° C. in a CO₂/air atmosphere (5%/95%, v/v) and 90% humidity, the medium being renewed every day. For the skin equivalents (SE), keratinocytes of human foreskin were sown in a density of 200,000 cells/cm² onto the 14-day-old DEs and incubated for another 7 days under submerse conditions in a medium consisting of 60% DMEM, 30% HAM F12 and 10% FCS supplemented with 25 μg/ml gentamycin, 100 Ul/ml penicillin, 1 μg/ml amphotericin B, 50 μg/ml sodium ascorbate, 4 mM L-glutamine, 10 ng/ml epidermal growth factor (EGF), 0.4 μg/ml hydrocortisone, 0.12 Ul/ml insulin, 10⁻⁹ M choleratoxin, 5 ng/ml transferrine and 180 μM adenine. The skin equivalents were then cultivated for another 14 days at the air/liquid interface in modified keratinocyte medium (DMEM-HAM F12 supplemented with 0.4 μg/ml hydrocortisone and 0.12 Ul/ml insulin).

By comparison with the monolayer cultures normally used, this model corresponds far better to the in vivo situation because keratinocytes and fibroblasts are in close contact with one another and, as in vivo, can exchange signal substances. In addition, the upper skin layers perform a filter function, for example for UVB rays.

2. Detection of MMP 1 Inhibition by Liposome-Encapsulated Photolyase

To detect MMP 1 inhibition by liposome-encapsulated photolyase, the skin models were first exposed to UVB radiation to generate the pyrimidine dimers. They were then exposed to UVA radiation to activate the photolyase so that this radiation could develop its effect on the repair of the keratinocyte DNA and on the inhibition of the MMP 1 in the fibroblasts.

2.1 Determination of the UVA Dose Required to Activate the Photolyase

It was known from the literature that a dose of 9 J UVA/cm² is sufficient to activate the photolyase. The UVA lamp used had an output of 1.7 mW/cm² so that an exposure time of 90 minutes was needed to obtain the photolyase activation dose.

2.2 Determination of Cell Activity After Combined UVB/UVA Irradiation

Another series of preliminary tests was conducted to determine what dose of the high-energy UVB radiation is tolerated by the cells of the skin equivalents. To this end, skin models were exposed first to various doses of UVB (varying from 100 to 800 mJ/cm², i.e. for an output of the UVB lamp used of 1.2 mW/cm², the exposure time was varied from 83 seconds to 11.1 minutes) and then to a dose of 9 J UVA/cm².

After the exposure, the skin models were incubated for 24 hours under standard conditions (37° C., 5% by vol. CO₂ and 90% humidity) in the nutrient medium of the air/liquid interface.

Finally, the vitality of the cells was determined by the MTT Test (procedure explained in 2.1.1). Table 1 shows the results of this vitality test. The vitality of the untreated control was used as reference (=100%) and all other measured values were related thereto.

TABLE 1
Cell vitality after combined UVB/UVA irradiation as measured by
the MTT Test (n = 2)

| | Relative vitality, based on non-irradiated |
|---|---|
| UVB dose applied [mJ/cm²] | skin models [%] |
| 0 | 100 |
| 100 | 78 |
| 200 | 82 |
| 800 | 79 |

The results show that ca. 80% of the cells are still vital after exposure to up to 800 mJ/cm² UVB. For the UVB irradiation of the skin models to produce pyrimidine dimers or for activation of the MMP 1 synthesis, a dose of 360 mJ UVB/cm² (=5 mins. exposure to UVB) was selected on the basis of the MTT test results, corresponding to an arithmetic mean value of the tested doses.

2.2.1 MTT Test for Determining Vitality

The MTT Test provides information on cell proliferation and cytotoxicity. In the test, the metabolic activity of living cells is determined. The tetrazolium salt 3-[4,5-dimethylthiazol-2-yl]-2,5-diphenyl tetrazolium bromide (MTT) is reduced in living cells and converted into a water-insoluble formazane salt. The formazane salt is extracted and photometrically quantified. The quantity of formazane salt formed is a measure of the number of living cells in the sample investigated. The exact test procedure is disclosed in J. Immunol. Methods 65, 55, 1983 (T. Mosmann) to which reference is explicitly made here.

To prepare the MTT solution, 2 ml of an MTT solution (conc. 1 mg MTT/ml in phosphate buffered saline=PBS) were pipetted into each well of a 24-well tray. The skin models were transferred to the tray and incubated for 3 hours at 37° C. in an atmosphere of CO₂/air (5%/95%, v/v) and 90% humidity. On completion of incubation, the skin models were transferred to centrifuge tubes and the formazane salt formed was extracted with 4 ml extractant (292 ml isopropanol+8 ml 1 M HCl) for 1.5 hours in a shaking machine. The optical density of an aliquot of 200 μl was measured in a 96-well plate at a wavelength of 540 nm (Titerek Multiscan MCC 340, Flow Laboratories).

2.3 Analysis of MMP 1 Inhibition

A test was conducted to determine to what extent the treatment of irradiated human skin equivalents with a cream formulation containing photolyase is able to reduce the synthesis of MMP 1 induced by exposure to UVB. To this end, human multilayer skin models were exposed to a UVB dose of 360 mJ/cm² and then treated with a cream containing 0.1% by weight Photosome™. In a control experiment, simultaneously UVB-irradiated skin models (b) were treated with a placebo cream with no Photosome™ or (c) remained untreated. To this end, quantities of 5 μl of cream according to the invention and placebo cream (corresponding to ca. 3.8 mg/cm²) were applied and carefully spread with a soft brush.

In another control experiment, the skin models remained unexposed and untreated. All skin equivalents were then incubated for 3 hours at 37° C. in an atmosphere of CO₂/air (5%/95%, v/v) and 90% humidity (standard conditions) in order to guarantee permeation of the active ingredient and were then exposed to a UVA dose of 9 J/cm² to activate the photolyase.

The skin models were incubated for another 48 hours under standard conditions. The RNA of the cells was then prepared by the method of R. E. Kingston et al. (1997), Preparation and Analysis of RNA in “Current Protocols in Molecular Biology”, Eds. F. M. Ausubel et al., John Wiley and Sons Inc., Chapter 4.

The expression of the MMP 1 gene was analyzed in a Northern Blot experiment. A radioactive gene probe specific to the mRNA of the MMP 1 was used for this purpose. The production of mRNA is the first and hence most important step in the synthesis of MMP 1. Substances which have an effect on mRNA production therefore also have an effect on the protein quantity and the enzyme activity of MMP 1.

Control experiments with a probe for the 18S—RNA showed that comparable quantities of RNA were analyzed. To quantify the Northern Blot signal intensities, the autoradiograms were densitometrically evaluated. The signals for MMP 1 were standardized to the associated values of the signals of the 18S—RNA.

These analysis methods are widely known among experts and are documented in particular in Brenneisen, P. et al. (1996), Photochem. Photobiol. 64, 877-885 and in Poswig, A. et al. (1999), J. Invest. Dermatol. 112, 13-18, to which reference is explicitly made here.

The standardized MMP 1 signal values for the exposed skin models not treated with cream were used as reference (=100%) and the values of the other skin models were related thereto (Table 3).

MMP 1 was determined in the following skin model samples:

sample 1: exposure to UVB, no treatment with cream

sample 2: exposure to UVB+treatment with placebo cream

sample 3: exposure to UVB+treatment with Photosome™ cream in accordance with the invention

sample 4: no exposure to UVB, no treatment with cream

TABLE 2
Composition of the test creams according to the invention

| | Lamellar cream, o/w emulsion |
|---|---|
| Ingredient | [% by weight] |
| Cetiol ® OE | 7.0 |
| Cetiol ® V | 7.0 |
| Lanette ® 22 | 7.0 |
| Lanette ® E | 0.18 |
| Baysilonöl M 350 | 0.5 |
| Vitamin E acetate | 1.0 |
| Retinyl palmitate | 1.0 |
| D-panthenol | 1.0 |
| Photosome ™ | 0.1 |
| Glycerol | 5.0 |
| Formalin solution (37%) | 0.08 |
| Water | to 100 |

TABLE 3
Quantity of UVB-induced MMP 1 as a function of the cream
treatment

| | Northern Blot signals for MMP 1, |
|---|---|
| | based on the signal for sample 1 [%] |
| 1 | 100 |
| 2 | 41 |
| 3 | 22 |
| 4 | 5 |

The treatment of the skin models with a cream formulation containing photolyase (sample 3) reduced the expression of MMP 1 by almost 80%.

The exposure of the human skin equivalents to UVA light corresponding to a dose of 9 J/cm² did not produce any significant induction of MMP 1 so that the measured effects were attributable solely to the UVB-induced synthesis of MMP 1.

The results of these analyses show that liposome-encapsulated photolyase is capable of effectively reducing the UVB-induced expression of MMP 1.

3. Other Formulation Examples

| | Example 3.1 | Example 3.2 |
|---|---|---|
| | o/w PIT emulsion | w/o emulsion |
| Ingredient | [% by weight] | [% by weight] |
| Cetiol ® OE | 7.5 | 7.0 |
| Cetiol ® V | 7.5 | 7.0 |
| Lanette ® O | 4.0 | — |
| Glyceryl palmitate | 2.2 | — |
| Eumulgin ® B 2 | 2.1 | — |
| Baysilonöl M 350 | 0.5 | — |
| Vitamin E acetate | 1.0 | — |
| Retinyl palmitate | 1.0 | 1.0 |
| Biotin | 0.005 | — |
| Dihydro-3-hydroxy-4.4-dimethyl- | 1.0 | 1.0 |
| 2(3H)-furanone (pantolactone) | | |
| Algal extract SPHM 3002 | — | 1.0 |
| Photosome ™ | 0.1 | 0.1 |
| Glycerol | 5.0 | 5.0 |
| MgSO₄.H₂O | — | 0.7 |
| Formalin solution (37%) | 0.08 | 0.08 |
| Water | to 100 | to 100 |

| | Example 3.3 | Example 3.4 | |
|---|---|---|---|
| | Lipoprotein | Glycolipid | |
| Ingredient | cream | cream | Example 3.5 |
| Montanov ® 202 | — | — | 4.0 |
| Thistle oil | 3.0 | — | — |
| Evening primrose oil | — | 3.0 | — |
| Myritol ® PC | 3.5 | 3.5 | — |
| Myritol ® 331 | — | — | 3.0 |
| Myritol ® 318 | — | — | 2.0 |
| Cetiol ® MM | — | 2.5 | — |
| Cetiol ® B | — | — | 7.0 |
| Cetiol ® SB 45 | — | — | 0.5 |
| Lanette ® 22 | 3.0 | — | — |
| Cutina ® GMS-V | 3.0 | 4.0 | 2.0 |
| Lanette ® O | 3.0 | 2.0 | 1.0 |
| Edenor ® IPS | 6.0 | 6.0 | — |
| Cosmacol ® PLG | — | 3.0 | — |
| Baysilonöl M350 | 1.0 | 1.0 | 0.5 |
| Eusolex ® 6300 | 0.6 | 0.6 | 3.0 |
| Parsol ® 1789 | 0.1 | 0.1 | 2.0 |
| Controx ® KS | 0.05 | 0.05 | 0.05 |
| pHB propyl ester | 0.2 | — | 0.2 |
| Photosome ™ | 0.1 | 0.1 | 0.1 |
| Panthenol | 1.0 | 1.0 | 1.0 |
| Herbasol ®, mallow distillate | — | 1.0 | — |
| Herbasol ®, rosemary extract | — | — | 1.0 |
| Dry Flo ® Plus | — | 3.0 | — |
| Hexane-1,6-diol | — | 6.0 | — |
| Dipropylene glycol | — | 5.0 | — |
| Glycerol | 5.0 | — | — |
| DSC-H N | — | 5.0 | — |
| V-Protein liquid | 9.0 | — | — |
| Tioveil ®-AQ-N | 2.0 | — | — |
| Citric acid | 0.1 | — | — |
| Sepigel ® 305 | 3.0 | 0.4 | — |
| Methocel ® E 4M | — | — | 0.2 |
| Herbasol ®, green tea | — | 1.0 | — |
| distillate | | | |
| Water | to 100 | to 100 | to 100 |

| | Example 3.6 |
|---|---|
| Ingredient | Mild cleaning gel |
| Eumulgin ® HRE 40 | 0.6 |
| Eucarol ® AGE-ET | 2.0 |
| 1,2-Propylene glycol | 10.0 |
| Photosome ™ | 0.1 |
| Bisabolol | 0.1 |
| D-Panthenol | 0.5 |
| pHB propyl ester | 0.1 |
| pHB methyl ester | 0.2 |
| Carbopol ® ETD 2020 (0.5%) | 50.0 |
| Water | to 100 |

| | Example 3.7 | Example 3.8 |
|---|---|---|
| Ingredient | Matrix plaster | Matrix plaster |
| DURO-TAK ® | 76 | 76 |
| Photosome ™ | 0.1 | — |
| Ultrasome ™ | — | 0.1 |
| Panthenol | 2 | — |
| Dihydro-3-hydroxy-4,4-dimethyl- | — | 2 |
| 2(3H)-furanone (pantolactone) | | |
| Herbasol ®, hibiscus distillate | 1 | — |
| Herbasol ®, green tea distillate | — | 1 |
| Aloe vera gel | 1 | 1 |
| Tioveil ®-AQ-N | 2 | — |
| Eusolex ® OCR | 1 | — |
| Propylene glycol monooleate | 5 | 5 |
| Controx ® KS | 0.05 | 0.05 |
| Water | to 100 | to 100 |

| Ingredients of the active | Example 3.9 | Example 3.10 |
|---|---|---|
| substance reservoir | Gel reservoir plaster | Gel reservoir plaster |
| Photosome ™ | 0.1 | — |
| Ultrasome ™ | — | 0.1 |
| Panthenol | 1.0 | — |
| Pantolactone | | 1.0 |
| Bisabolol | 1.0 | 1.0 |
| Herbasol ®, | — | 1.0 |
| hibiscus distillate | | |
| Ethanol | 40 | 40 |
| Mowiol ®18-88 | 8.0 | 8.0 |
| Luviskol ®K 80 | 5.0 | 5.0 |
| Controx ®KS | 0.05 | 0.05 |
| Brij ®-35 | 2.0 | 2.0 |
| Cremophor ® CO-40 | 0.5 | 0.5 |
| Glycerol | 5.0 | 5.0 |
| Water | to 100 | to 100 |

Raw materials used:

| Name | INCI |
|---|---|
| Algal extract SPHM 3002 | Aqua, Algae (Linne) |
| Aloe vera gel (Provital SA): | |
| 0.85-1.55% by weight active substance in | Aloe Barbadensis (Linne) |
| propylene glycol/water | |
| Baysilonöl M 350 | Polydimethylsiloxane/Dimethicone |
| Brij ®-35 | Laureth-23 |
| Carbopol ® ETD 2020 (0.5%) | Acrylates/C10-30 Alkyl Acrylate |
| | Crosspolymer |
| Eumulgin ® B 2 | Ceteareth-20 |
| Cetiol ® B | Dibutyl Adipate |
| Cetiol ® MM | Myristyl Myristate |
| Cetiol ® SB 45 | Butyrospermum Parkii (Linne) |
| Controx ® KS: | Tocopherol, Hydrogenated Palm Glycerides |
| | Citrate |
| Cosmacol ® PLG | Tri-C12-13 Alkyl Citrate |
| Cremophor ® CO-40 | PEG-40 Hydrogenated Castor Oil |
| Cutina ® GMS (C₁₆₋₁₈ fatty acid | Glyceryl Stearate |
| mono/diglyceride | |
| Cetiol ® V | Decyl Oleate |
| Cetiol ® OE | Dicaprylylether |
| Dry Flo ® Plus | Aluminium Starch Octenylsuccinate |
| DSC-H N (ex Exsymol) | Dimethylsilanol Hyaluronate |
| DURO-TAK ® (National Starch and Chemical): | Polyacrylate Copolymer |
| ca. 50% acrylate copolymer in spirit/ethyl- | |
| acetate/methanol/ethanol | |
| Eucarol ® AGE-ET UP (30% active substance | Sodium Cocopolyglucose Tartrate |
| in water) | |
| Eumulgin ® HRE 40 | PEG-40 Hydrogenated Castor Oil |
| Eusolex ® 6300 | 4-Methylbenzylidene Camphor |
| Eusolex ® OCR | Octocrylene |
| Herbasol ®, hibiscus distillate (Cosmetochem) | Water, Alcohol denat., Althea officinalis |
| Herbasol ®, green tea distillate | Water, Camellia sinensis extract |
| Herbasol ®, mallow distillate (Cosmetochem) | Aqua, SD Alcohol 39-C, Malva Sylvestris |
| | (Linne) |
| Herbasol ®, rosemary extract | Water, Propylene Glycol, Rosmarinus |
| | officinalis |
| Edenor ® IPS | Isopropyl Stearate |
| Lanette ® E | Sodium Cetearyl Sulfate |
| Lanette ® O | Cetearyl Alcohol |
| Lanette ® 202 | Behenyl Alcohol |
| Lifidrem ® PPST-GHK-4 (Coletica): pea protein | Pea Extract (Pisum Sativum (Linne)), |
| extract/C₁₆₋₁₈ fatty acid condensate | Sodium Stearate, Sodium Chloride |
| Methocel ® E 4M | Hydroxypropyl Methylcellulose |
| Montanov ® 202 | Arachidyl Alcohol, Behenyl Alcohol, |
| | Arachidyl Glucoside |
| Myritol ® 318 | Caprylic/Capric Triglyceride |
| Myritol ® 331 | Cocoglycerides |
| Myritol ® PC | Propylene Glycol Dicaprylate/Dicaprate |
| Evening primrose oil | Evening Primrose Oil Oenothera Biennis |
| | (Linne) |
| Parsol ® 1789 | Butyl Methoxydibenzoylmethane |
| pHB propyl ester | Propylparaben |
| Photosome ™ | Plankton Extract and Lecithin |
| Mowiol ® 18-88 | Polyvinyl alcohol, partly saponified |
| Luviskol ® K 80 | Polyvinyl pyrrolidone |
| Sepigel ® 305 | Polyacrylamide, C13-14 Isoparaffin, Laureth- |
| | 7 |
| Tioveil ®-AQ-N (Uniqema):titanium dioxide | Cl 77891 (Titanium Dioxide), Alumina, Silica, |
| dispersion | Sodium Polyacrylate |
| Ultrasome ™ | Micrococcus lysate |
| V-Protein liquid COS 152/22 A (Cosmetochem) | Aqua, Propylene Glycol, Hydrolyzed Pea |
| | Protein (Pisum Sativum) |
| Vitamin E acetate | Tocopheryl Acetate |

What is claimed is:

1. A method for inhibiting the light-induced degradation of collagen in human skin comprising applying to the skin a composition containing DNA repair enzymes.

2. The method of claim 1 wherein the DNA repair enzyme is photolyase.

3. The method of claim 1 wherein the DNA repair enzyme is T4 endonuclease V.

4. The method of claim 1 wherein the DNA repair enzyme is a mixture of photolyase and T4 endonuclease V.

5. The method of claim 1 wherein the composition is applied as a preventive treatment.

6. The method of claim 1 wherein the DNA repair enzyme(s) is/are used in a quantity of 1·10⁻⁶ to 5·10⁻²% by weight, based on the composition as a whole.

7. A method for inhibiting the expression of the activity of the matrix metal proteinase MMP 1 in human skin comprising applying to the skin a composition comprising DNA repair enzymes.

8. The method of claim 7 wherein the DNA repair enzyme is photolyase.

9. The method of claim 7 wherein the DNA repair enzyme is T4 endonuclease V.

10. The method of claim 7 wherein the DNA repair enzyme is a mixture of photolyase and T4 endonuclease V.

11. The method of claim 7 wherein the composition is applied as a preventive treatment.

12. The method of claim 7 wherein the DNA repair enzyme(s) is/are used in a quantity of 1·10⁻⁶to 5·10⁻²% by weight, based on the composition as a whole.

13. The method of claim 7 wherein the inhibition occurs in the dermis of the skin.

14. A cosmetic or pharmaceutical skin treatment composition comprising photolyase and/or T4 endonuclease V further comprising a compound selected from the group consisting of
a) at least one substance selected from the vitamins, provitamins or vitamin precursors of the vitamin B group or derivatives thereof and derivatives of 2-furanone,
b) at least one plant extract, and
c) at least one other MMP 1-inhibiting substance selected from propyl gallate, precocenes, 6-hydroxy-7-methoxy-2,2-dimethyl-1 (2H)-benzopyran, 3,4-dihydroxy-6-hydroxy-7-methoxy-2,2-dimethyl-1 (2H)-benzopyran and mixtures thereof.

15. The composition of claim 14 further comprising at least one ester of retinol (vitamin A₁) with a C₂₋₁₈ carboxylic acid.

16. The composition of claim 14 further comprising at least one ionic surfactant.

17. The composition of claim 14 further comprising at least one nonionic surfactant with an HLB value of 8 or lower.

18. The composition of claim 14 further comprising at least one organic or mineral or modified mineral UV filter.

19. The composition of claim 14 further comprising at least one protein hydrolyzate and/or derivative thereof.

20. The composition of claim 14 further comprising at least one amino acid selected from glycine, serine, threonine, cysteine, asparagine, glutamine, pyroglutamic acid, alanine, valine, leucine, isoleucine, proline, tryptophane, phenylalanine, methionine, aspartic acid, glutamic acid, lysine, arginine and histidine and also the zinc salts and the acid addition salts of these amino acids.

21. The composition of claim 14 further comprising at least one mono-, oligo- or polysaccharide and/or derivatives thereof.

22. The composition of claim 14 further comprising at least one polymer selected from the group consisting of film-forming polymers, emulsion-stabilizing polymers, thickening polymers and adhesive polymers.

22. The composition of claim 13 wherein the at least one substance selected from the vitamins, provitamins or vitamin precursors of the vitamin B group are panthenol, pantolactone, nicotinic acid amide or biotin.
